(19) United States
(12) Patent Application Publication
(10) Pub. No.: US 2006/0175604 A1
(43) Pub. Date: Aug. 10, 2006

(54) Novel type of attachment of organic molecules to a silicon surface for producing memory elements having organic constituents

(76) Inventor: Klaus-Dieter Ufert, Unterschleissheim (DE)

Correspondence Address: JENKINS, WILSON, TAYLOR & HUNT, P. A., 3100 TOWER BLVD, SUITE 1200, DURHAM, NC, 27707, US

(21) Appl. No.: 11/336,107

(22) Filed: Jan. 20, 2006

(30) Foreign Application Priority Data
Jan. 25, 2005 (DE) 102005003475.6

(51) Int. Cl. H01L 29/08; C07D 213/84

(52) U.S. Cl. 257/40; 546/255; 546/286; 977/943

(57) ABSTRACT

A nonvolatile memory element in a semiconductor structure comprises at least two contacts, of which one comprises silicon, and a monolayer of conductive organic molecules producing a conductive connection between the two contacts. The organic molecule comprise alternating ethynyl and aryl groups and have at least one group selected from among benzonitrile, phenylacetonitrile, benzoylacetonitrile, benzoyl cyanide by means of which the molecule bonds via the cyano group to the silicon contact at the end of the molecule.

### BACKGROUND OF THE INVENTION

1. Field of the Invention

The present invention relates to a nonvolatile memory element in which organic molecules are bound via a novel type of end group to silicon surfaces, in particular via the nitrogen of a nitrile group to a silicon surface or to a nitrided silicon surface.

2. Description of the Related Art

Since miniaturization of semiconductor elements is progressing continually and the classic methods of etching structures will come up against foreseeable limits, new types of structures have to be developed. A promising approach is the use of organic molecules in semiconductor elements. For example, conjugated oligophenyleneethynylene can be used as molecular wires in components (with nonlinear I(V) curves or for dynamic random access memories (DRAMs)).

The memory concepts used hitherto (floating gate memories such as flash and DRAM) are based on the storage of charges in inorganic, silicon-based materials. In contrast thereto, organic molecules such as amino-4-ethynylphenylbenzenethiol which grow in a self-assembled molecular monolayer on a layer of gold can effect the storage of information according to the principle of resistive switching between two stable resistances in the organic molecules.

The preparation of oligophenyleneethynylenes by means of known organic synthetic methods is described in the literature. Thus, for example, Moroni et al. (Macromolecules 1997, 30, 1964-1972) describe the coupling of acetylene derivatives with phenyl derivatives in the presence of platinum or palladium as catalyst. The preparation of organic molecules for use in semiconductor elements is also described by Tour et al. (Chem. Eur. J. 2001, 7, No. 23, 5118-5134).

The construction of a test setup is described by Chen et al. in Science, Vol. 286, 1550-1552. In the latter publication and also in Reed et al. (Applied Physics Letters, Vol. 78, No. 23, 3735-3737) and Chao Li et al. (Applied Physics Letters, Vol. 82, No. 4, 645-647), metal contacts, preferably gold, are used as the surface to which organic molecules are applied. Application is carried out using the mechanism of self-assembled monolayers (SAMs). Here, the molecules are bound to the gold surface by means of the sulphur via a thiol unit. A top contact is subsequently applied to the layer of organic molecules. This represents the second contact for the molecules. The organic molecules thus form a connection, viz. a molecular wire, between the two metal contacts.

An important problem is the bonding of an organic molecule to a substrate. Hitherto, the organic molecules have been attached only by means of thiol (SH) groups to a substrate layer of gold. Here, the adhesion of the molecule to the metal, in particular, is often poor. The organic molecules are applied to the substrate in the form of self-assembled molecular monolayers. They are anchored via the end or attachment group of the molecule. The binding energy is often low and bonding is seldom chemically covalent. In addition, the adhesion is greatly dependent on the morphology of the substrate. The conductivity behaviour and the current-voltage characteristics of such organic molecular semiconductor elements are therefore often not reproducible and vary greatly with the properties of the unsatisfactory contact between the organic molecule and the substrate.

The end group is responsible for the chemical bonding and the mechanical adhesion of the organic molecule to the substrate. If the chemical and mechanical contact is not optimal, electron transfer (the electrical conductivity) via this molecule-substrate bridge is adversely affected. A measure of this is the conductivity gap.

However, good coupling to a silicon substrate is important for integration into the existing CMOS technology. This cannot be achieved satisfactorily in chemical and electrical terms by means of the thiol groups used at present. The resistive memory states are therefore not controllable and cannot be optimally set. It would be advantageous for the additional process step of application of a metal layer to be able to be omitted.

### SUMMARY OF THE INVENTION

It is an object of the present invention to make coupling of the organic molecules directly to a silicon substrate possible.

The principle underlying the present invention is the attachment of organic molecules via the nitrogen of a nitrile group directly to a substrate composed of silicon. Both chemical bonding and electrical bonding between molecule and substrate are improved in this way. The gold contact layer which is obligatory in the prior art thus becomes superfluous.

The organic molecule acts as a conductive connection between two contacts. According to the invention, one of the two contacts is a silicon substrate. The organic molecule is built up of alternate ethynyl and aryl groups. A cyano group is present at one end of this chain to allow attachment of the organic molecule to the substrate. This cyano group is part of a group selected from among benzonitrile, phenylacetonitrile, benzoylacetonitrile and benzoyl cyanide.

In a preferred embodiment, at least one electron-pulling group is present on at least one aryl group. This electron-pulling group can be, for example, NH₂ or NO₂. In a further embodiment, one or more of the carbon atoms of the aryl group can be substituted so as to produce an electron-deficient aryl group. In another embodiment, one or more of the aryl groups can be replaced by a substituted or unsubstituted pyridine.

The group via which the organic molecule bonds to the silicon contact may preferably be benzonitrile or phenylacetonitrile.

The organic molecule having alternating ethynyl and aryl groups can be described by the formula I below: 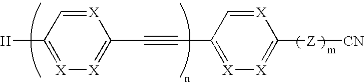
where

n is an integer from 0 to 10,

m is 0 or 1,

Z is a linker group which can be CH₂, CO or COCH₂, and the radicals X can each be, independently of one another in each molecule, C—H, N or C—Y, where Y is an electron-pulling group.

In the formula I above, Y is preferably an NH₂ group or an NO₂ group. Z is preferably a CH₂ group or m is preferably 0. The number of alternating ethynyl and aryl groups is determined by the number n. This is preferably in the range from 1 to 5 and is more preferably 2, 3 or 4.

In a preferred embodiment, the silicon contact is a nitrided silicon surface. The second contact, known as the top contact, is preferably a palladium- or titanium-coated carbon nanotube or a gold top contact. The latter can, for example, be applied under very mild conditions by vapour deposition of a gold layer under reduced pressure with cooling of the substrate.

A further aspect of the invention relates to a process for producing a nonvolatile memory element. Here, in parts, a silicon surface is provided and organic molecules comprising alternating ethynyl and aryl groups and having at least one group selected from among benzonitrile, phenylacetonitrile, benzoylacetonitrile and benzoyl cyanide by means of which the molecules bond via the cyano group to the silicon surface at the end of the organic molecules are applied to the silicon surface by bringing the silicon surface into contact with the molecules by the method of deposition of self-assembled monolayers (SAMs) so that the molecules bond to the silicon surface via the cyano group and a top contact is subsequently applied.

In a preferred example, the organic molecules are applied to a nitrided silicon surface.

Examples which illustrate the invention are depicted in the drawings and explained in more detail in the following description.

### DESCRIPTION OF THE FIGURES

FIG. 1 shows a reaction scheme for preparing 2′-amino-4-ethynylphenyl-4′-ethynylphenyl-5′-nitro-1-benzonitrile.

FIG. 2 shows a schematic depiction of the structure of a nonvolatile memory element having organic molecules according to the invention.

### DESCRIPTION OF THE PREFERRED EMBODIMENTS

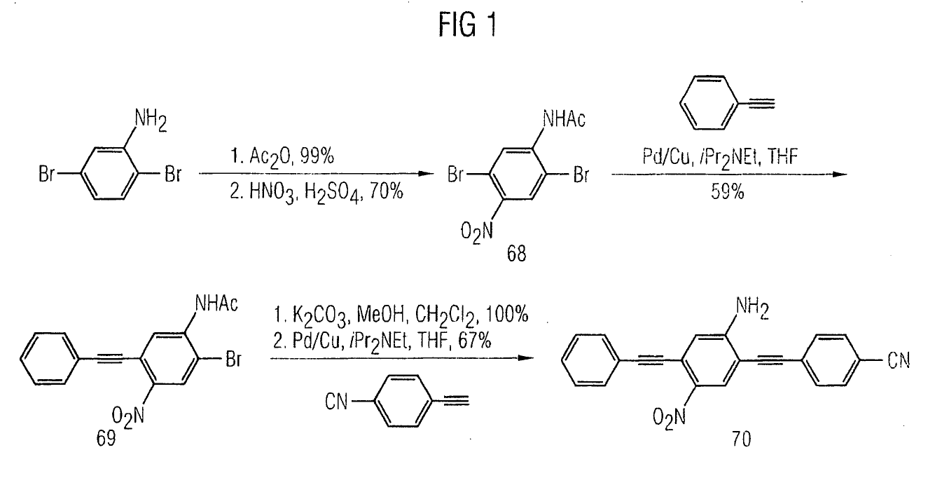

The reaction sequence for preparing 2′-amino-4-ethynylphenyl-4′-ethynylphenyl-5′-nitro-1-benzonitrile is shown schematically in FIG. 1. The synthesis of 2,5-dibromo-4-nitroacetanilide is described in detail by Moroni et al. (Macromolecules 1997, 30, 1964-1972). 2,5-Dibromo-4-nitroacetanilide is coupled with phenylacetylene in the presence of a Pd/Cu catalyst. The protective acetyl group is subsequently removed by action of potassium carbonate and methanol. This intermediate is finally coupled with the end group containing the nitrile in the presence of a Pd/Cu catalyst to give the end product. Further basic building blocks which display resistive memory properties are 4,4′-di(ethynylphenyl)-1-benzene and 4,4′-di(ethynylphenyl)-2′-nitro-1-benzene.

The silicon electrode structure is produced on the basis of the SOI technology. Here, an oxidized Si wafer is pressed together with a pure Si wafer under high pressure at high temperature and the Si wafer side is subsequently thinned by means of CMP. The etching of the Si tracks can be carried out using present-day Si etching techniques such as dry etching by means of reactive plasma etching based on HBr and O₂ or SF₆.

To apply the organic molecule prepared by the above method, the Si substrate is dipped into the solution containing the organic molecule. After deposition, the substrate is rinsed and dried in a stream of dry N₂ gas.

The basis of the example is a p-Si (100) wafer which is provided with functional CMOS technology elements corresponding to the prior art.

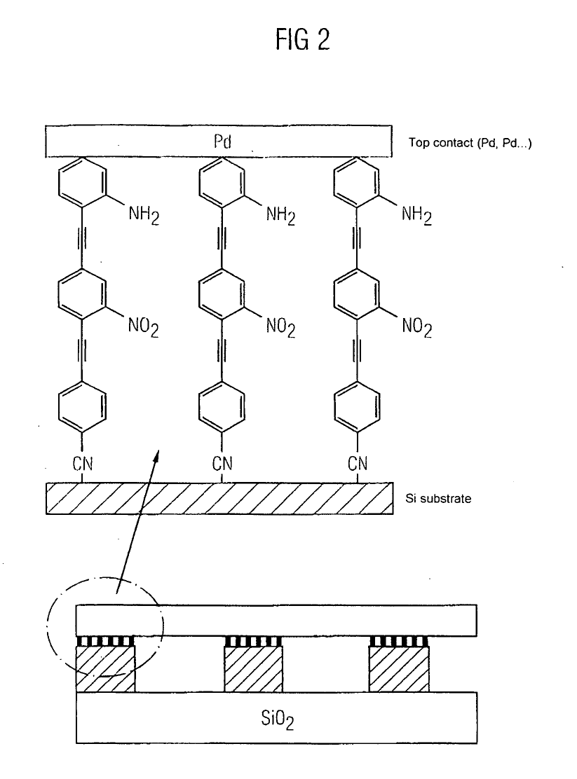

The application of such an organic molecule to a silicon substrate is shown in FIG. 2. Here, the surface can be structured by means of classic methods. The application is carried out by means of the self-assembled monolayer technology. Palladium is used as top contact.

Although modifications and changes may be suggested by those skilled in the art, it is the intention of the inventor to embody within the patent warranted heron all changes and modifications as reasonably and properly come within the scope of his contribution to the art.

I claim as my invention:

1. A nonvolatile memory element in a semiconductor structure, comprising:
at least two contacts, of which one comprises silicon; and
a monolayer of conductive organic molecules producing a conductive connection between said two contacts; said organic molecule comprising alternating ethynyl and aryl groups and having at least one group selected from among benzonitrile, phenylacetonitrile, benzoylacetonitrile, benzoyl cyanide by means of which said molecule bonds via said cyano group to said silicon contact at the end of said molecule.

2. The memory element of claim 1, wherein at least one of said aryl groups is substituted by an electron-withdrawing group.

3. The memory element of claim 2, wherein said electron-withdrawing group is NH₂ or NO₂.

4. The memory element of claim 1, wherein at least one of said aryl groups is a substituted or unsubstituted pyridine.

5. The memory element of claim 1, wherein said group which bonds to said silicon contact is benzonitrile or phenylacetonitrile.

6. The memory element of claim 1, wherein said silicon contact is a nitrided silicon surface.

7. The memory element of claim 1, wherein said organic molecule has the formula I below: 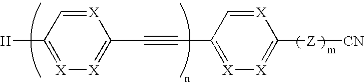
wherein
n is an integer from 0 to 10,
m is 0 or 1,
Z is a linker group which can be CH₂, CO or COCH₂, and
X can each be, independently of one another in each molecule, C—H, N or C—Y, where Y is an electron-withdrawing group.

8. The memory element of claim 7, wherein said electron-withdrawing group Y is NH₂ or NO₂.

9. The memory element of claim 7, wherein Z is a CH₂ group or m=0.

10. The memory element of claim 7, wherein n is in the range from 1 to 5.

11. The memory element of claim 7, wherein n is 2, 3 or 4.

12. The memory element of claim 1, wherein said second contact is a palladium-coated carbon nanotube or gold.

13. The memory element of claim 7, wherein said second contact is a palladium-coated carbon nanotube or gold.

14. A method for producing a nonvolatile memory element, on which, in parts, a silicon surface is provided and organic molecules comprising alternating ethynyl and aryl groups and having at least one group selected from among benzonitrile, phenylacetonitrile, benzoylacetonitrile and benzoyl cyanide by means of which the molecules bond via the cyano group to the silicon surface at the end of the molecules are applied to the silicon surface by bringing the silicon surface into contact with said molecules by the method of deposition of self-assembled monolayers so that said molecules bond to said silicon surface via said cyano group and a top contact is subsequently applied.

15. The method of claim 14, wherein said silicon surface to which said organic molecules are applied is nitrided.
